(19) United States
(12) Patent Application Publication
(10) Pub. No.: US 2022/0139562 A1
(43) Pub. Date: May 5, 2022

(54) USE OF VIRTUAL AGENT TO ASSESS PSYCHOLOGICAL AND MEDICAL CONDITIONS

(71) Applicant: MODALITY.AI, INC., San Francisco, CA (US)

(72) Inventors: Michael Neumann, Waiblingen (DE); Oliver Roesler, Weyhe (DE); David Suendermann-Oeft, San Francisco, CA (US); Vikram Ramanarayanan, Berkeley, CA (US)

(21) Appl. No.: 17/471,929

(22) Filed: Sep. 10, 2021

Related U.S. Application Data
(60) Provisional application No. 63/050,284, filed on Jul. 10, 2020.

(51) Int. Cl. G16H 50/30; G16H 70/20; G10L 15/18

(52) U.S. Cl. CPC G16H 50/30; G10L 15/1815; G16H 70/20

(57) ABSTRACT

A virtual agent converses with a patient or other person to assess one or more psychological or other medical conditions of the other person. The virtual agent uses both semantic and affect content from the person to branch the conversation, and to assess a psychological or other condition of the person. The virtual agent can have artificial intelligence functionalities or can utilize a separate artificial intelligence functionality. A communication agent can be utilized to monitor a telecommunication session with the person, and if appropriate, modify relative bandwidth utilization between the audio and image inputs. The virtual agent/artificial intelligence agent can simultaneously assist multiple virtual agents, who are each conversing with a responding person assessing their psychological or other medical condition(s), in parallel. The contemplated virtual agents can be especially useful in assessing disorder severity in multiple neurological and mental disorders.

This application claims priority to provisional patent application Ser. No. 63/050284, filed on Jul. 10, 2020. The provisional and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling.

### FIELD OF THE INVENTION

The field of the invention is healthcare informatics, especially analysis of psychological or other medical conditions.

### BACKGROUND

The following description includes information that may be useful in understanding the present invention. It is not an admission that any of the information provided herein is prior art or relevant to the presently claimed invention, or that any publication specifically or implicitly referenced is prior art.

Diagnosis, detection, and monitoring of medically-related conditions remain a critical need. The problems are often exacerbated by: (i) lack of access to neurologists or psychiatrists; (ii) lack of awareness of a given condition and the need to see a specialist; (iii) lack of an effective standardized diagnostic or endpoint for many of these health conditions; (iv) substantial transportation and cost involved in conventional or traditional solutions; and in some cases, (v) shortage of medical specialists in these fields.

There have been many efforts to address these problems, including use of telemedicine, in which a practitioner interacts with a patient or patients utilizing telecommunications. Telemedicine does not, however, resolve problems associated with insufficient numbers of trained practitioners, or available time of existing practitioners. Psychological conditions, in particular, can often require lengthy times spent with responding patients. Current systems for telemedicine also fail to address inadequacies in electronic communications, especially in rural areas where adequate line speed and reliability are lacking.

As used herein, the term “patient” means any person with which a human or virtual practitioner is communicating with respect to a psychological or other condition, or potential such conditions, even if the person has not been diagnosed, and is not under the care of any practitioner. Where communication is via telecommunications, such person is also from time to time herein referred to as a “user”.

As used herein, the term “practitioner” broadly refers to any person whose vocation involves diagnosing, treating, or otherwise assisting in assessing or remediating psychological and/or other medical issues. In this usage, practitioners are not limited to medical doctors or nurses, or other degreed providers. Still further, as used herein, “medical conditions” should be interpreted as including psychological conditions, regardless of whether such conditions have any underlying physical etiology.

As used herein, the terms “assessment”, “assessing”, and related terms means weighing information from which at least a tentative conclusion can be drawn. The at least tentative conclusion need not rise to the level of a formal diagnosis.

As used herein, the term “virtual agent” broadly refers to a computer or other non-human functionality configured to operate as a practitioner in assessing or remediating psychological and/or other medical issues.

In view of the challenges mentioned above, there is a need for a virtual agent that can assess one or more psychological and/or other medical conditions of a patient or other user, utilizing both semantic and affect content. There is also a need for a communication agent that can cooperate with a practitioner and/or virtual agent to individually compensate for adverse telecommunications environments encountered during assessment sessions.

### SUMMARY OF THE INVENTION

The inventive subject matter provides apparatus, systems, and methods in which a virtual agent converses with a responding person to assess one or more psychological or other medical conditions of the user. The virtual agent uses both semantic and affect content from the responding person to branch the conversation, and also to interact with a data store to provide an assessment of the medical or psychological condition.

As used herein, the term “semantic content” means language information that a person is conveying, whether with verbalized words, with sign language, or with other body movements. Body movements used to convey semantic content can include facial expressions, gestures, postures, vocal intonations, and so forth. As a simple example, a person could answer a question with an audible “I don't know”, or simply shrug to convey “I don't know”. Either way, the semantic content is that the person doesn't know.

As used herein, the term “affect content” means the observable manifestations of an emotion. Emotions can also be gleaned from such manifestations as facial expressions, gestures, postures, vocal intonations, and so forth. Affect content can signal any emotion, including for example, anger, happiness, boredom, and frustration. In the example above, a person could unemotionally provide the semantic content that he/she does not know the answer to a question, and could alternatively provide that same semantic content, along with an angry facial expression, indicating the affect content of anger or frustration.

In other aspects, a communication agent monitors a telecommunication session with a user, and if appropriate, modifies relative bandwidth utilization between the audio and image inputs. Such modification can be advantageously based at least in part on at least one of the semantic and affect contents. For example, if communication speeds are low, and the responding person is mumbling, but is otherwise communicating with little affect, the communications agent might divert a greater bandwidth to the audio communication, and a lesser bandwidth to the video communication.

In still other aspects, the communications agent could be configured to modify relative bandwidth utilization between audio and image inputs, based at least in part on content of at least one of the questions being asked, rapidity of the user's speech or movement of a hand or body part.

In still other aspects, an artificial intelligence agent can assist the virtual agent in assessing the psychological or other medical condition(s) of the user.

In still other aspects, an artificial intelligence agent can simultaneously assist multiple virtual agents, who are each conversing with a responding person and assessing their psychological or other medical condition(s), in parallel.

Although it is contemplated that a virtual agent could rely solely on information from the responding person and the data store to assess the psychological or other medical condition(s) of the user, it is contemplated that the virtual agent could also make assessments with direct or indirect input from a human assessor, and/or from an artificial intelligence agent. In preferred embodiments, artificial intelligence agents would cooperate with multiple virtual agents and multiple human assessors to improve future assessments. Depending on the system architecture, the virtual agent, communications agent, and artificial intelligence agent can be entirely separate, or alternatively can overlap to any suitable degree.

Because of the focus on both semantic and affect contents, it is contemplated that the apparatus, systems, and methods disclosed herein can be especially useful in assessing disorder severity in multiple neurological and mental disorders. Specific examples include Parkinson's disease, schizophrenia, depression and autism spectrum disorder.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1 is a schematic view of a practitioner and/or a virtual agent conducting an assessment session with a responding person through electronic means.

FIG. 2A is a perspective view of an assessment session according to FIG. 1, in which the responding person has depression.

FIG. 2B is a perspective view of an assessment session according to FIG. 1, in which the responding person has Parkinson's disease.

FIG. 2C is a perspective view of an assessment session according to FIG. 1, in which the responding person has schizophrenia.

FIG. 2D is a perspective view of an assessment session according to FIG. 1, in which the responding person has bipolar disorder.

FIG. 2E is a perspective view of an assessment session according to FIG. 1, in which the responding person has autism spectrum disorder.

FIG. 3 is a flowchart of a virtual agent and other functionalities of cloud 110 interacting with a responding person, using both semantic content and affect content to assess a condition of the responding person.

### DETAILED DESCRIPTION

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention. Unless a contrary meaning is explicitly stated, all ranges are inclusive of their endpoints, and open-ended ranges are to be interpreted as bounded on the open end by commercially feasible embodiments.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

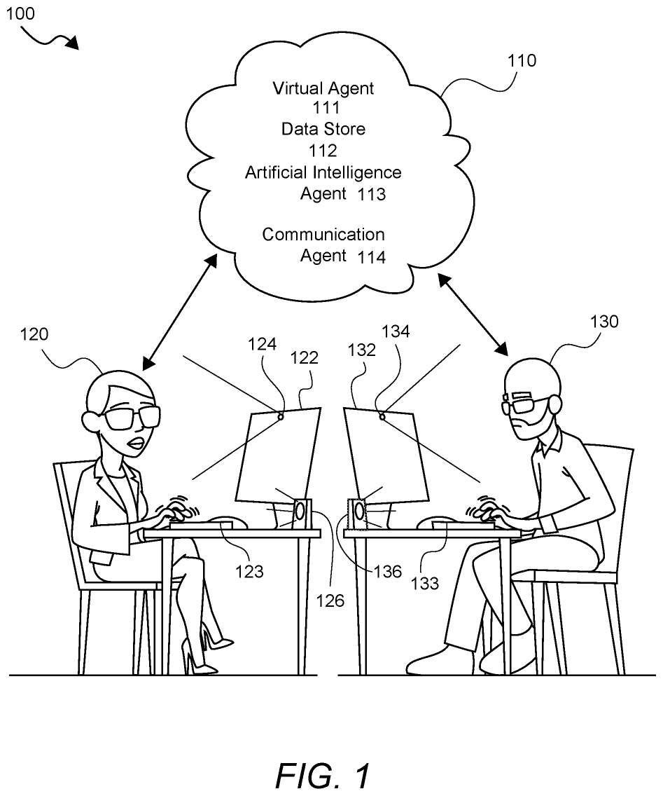

FIG. 1 is a schematic view 100 of a practitioner 120 and/or a virtual agent conducting an assessment session with a responding person 130 through electronic means 110.

Practitioner 120 is using a computer 122 having an optional keyboard 123, a combination camera/microphone 124, and a speaker 126. Although the computer is depicted as a desktop model, the computer and its other electronic components should be viewed generically to include any device or devices fulfilling the usual functions of these components, including for example a laptop, an iPad™ or other tablet, and even a cell phone.

Data processing and storage functionality (depicted here as cloud 110) should be viewed generically as one or more computing and storage devices that collectively operate to execute the functions of a virtual agent 111, a data store 112, an artificial intelligence agent 113, and a communication agent 114, including storing and executing instructions stored on a computer readable tangible, non-transitory medium. For example, contemplated computing and storage devices include one or more computers operating as a web server, database server, or other type of computer server, and related storage devices, and can be physically local to one another, or more likely are distributed in different cities and even different countries. Accordingly, practitioner 120 and responding person 130 might be in different parts of the same building, or widely separated across the planet. One should also appreciate that such servers and storage devices can be re-configured from time to time to produce better conversational responding person experiences, and more reliable assessment accuracy.

It should be appreciated that virtual agent, data store, artificial intelligence agents, and communication agent are depicted within cloud 110 without clear boundaries. This is done intentionally to show that these items are not necessarily separate. For example, functionalities of the virtual agent might well be combined with those of the artificial intelligence agent and/or the communications agent, whether or not the corresponding software or firmware is physically operating from the same hardware.

Responding person 130 is also using a computer 132 having an optional keyboard 133, a combination camera/microphone 134 that provides inputs to the practitioner 120/virtual agent 111/artificial intelligence agent 113, and a speaker 136. Computer 122 might or might not be similar in features to computer 132, and here again, computer 132 should be viewed generically to include any device or devices fulfilling the usual functions of these components, including for example a laptop, an iPad™ or other tablet, and even a cell phone.

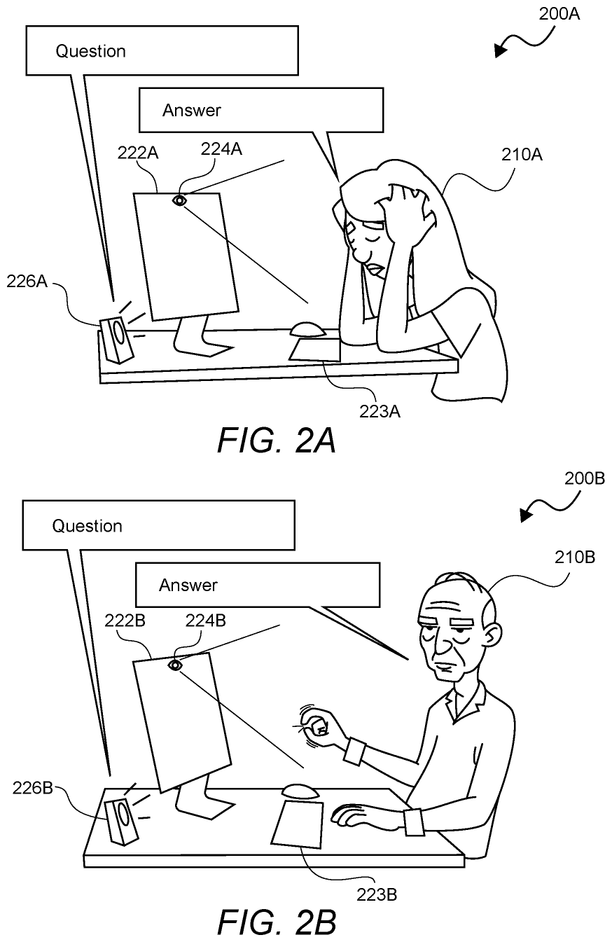

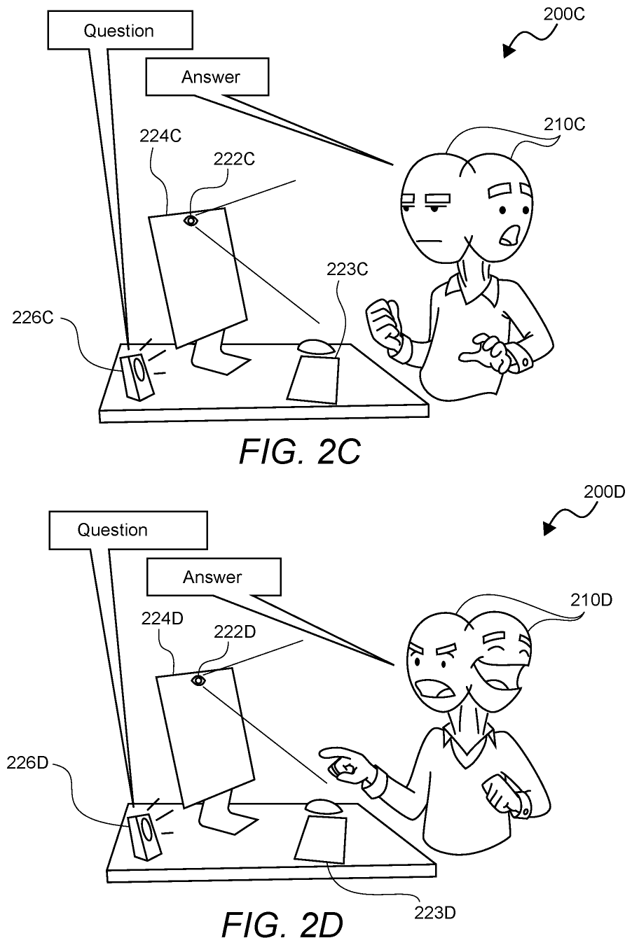

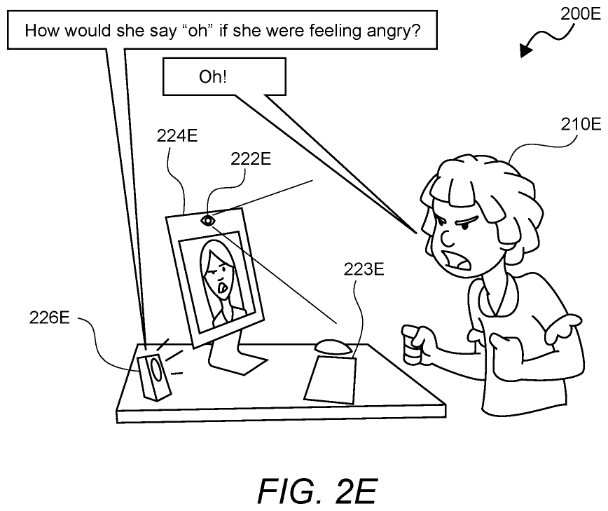

Practitioner 120 and responding person 130 are each depicted as sitting at a desk, however, it is contemplated that either or both of them could be interacting in any suitable posture, including for example, walking about, sitting on a couch, or lying in bed. Similarly, although practitioner 120 is shown as a middle aged woman, and responding person 130 is shown as an older man, FIG. 1 (and indeed FIGS. 2A-2E) should be viewed broadly enough to include all realistic ages and genders.

It should be appreciated that although practitioner 120 and responding person 130 should be viewed as sufficiently distant from one another that it is reasonable for them to be communicating through cloud 110.

As indicated above, FIG. 1 depicts an entity, whether practitioner 120 and/or virtual agent, conducting an assessment session with a responding person 130. In preferred embodiments the virtual agent will either be conducting the assessment session without concurrent presence of practitioner 120, or with practitioner 120 merely observing the session, and interacting if needed. This allows one or more instances of the virtual agent to concurrently assess multiple responding persons, who might in fact be situated hundreds or thousands of miles apart.

FIG. 2A is a perspective view of an assessment session 200A, in which the responding person 210A has depression. Question bubble should be interpreted as multiple questions and comments coming from practitioner 120 and/or the virtual agent 111/AI agent 113, and the answer bubble should be interpreted as multiple answers and other audible responses coming from the responding person 210A. A computer 222A operates an optional keyboard 223A, a combined camera/microphone 224A, and a speaker 226A.

Guidance regarding suitable questions and comments to assess depression can be taken from the priority provisional application, and the relevant literature. Following is an example of a very short portion of a possible assessment.
Agent: “Tell me more about your day. Are you having difficulty sleeping”?
Responding person: “I feel anxious all the time. I don't know how many bottles of wine I had last night. Terrible”.

In this example the virtual agent 111/AI agent 113 would utilize the speaker 226A to present the comment and question, and the responding person 210A would answer with the audible response and images coming through the combined camera/microphone 224A. The virtual agent/AI agent, in cooperation with the data store 112, would then analyze the semantic content of the spoken words, as well as the affect content provided by the tone of voice and facial expressions, to assist in assessing depression. In that way, both the semantic content and the affect content would be utilized to provide an assessment of a medical or psychological condition.

FIG. 2B is a perspective view of an assessment session 200B, in which the responding person 210B has Parkinson's disease. As with FIG. 2A, the question bubble should be interpreted as multiple questions and comments coming from practitioner 120 and/or the virtual agent 111/AI agent 113, and the answer bubble should be interpreted as multiple answers and other audible responses coming from the responding person 210B. A computer 222B operates an optional keyboard 223B, a combined camera/microphone 224B, and a speaker 226B.

Here again, guidance regarding suitable questions and comments can be taken from the priority provisional application, and the relevant literature. Following is an example of a very short portion of a possible assessment.
Agent: “Please perform a pitch glide. In other words, please start with/i/and move higher in pitch, like this” <agent demonstrates>
Responding person: <performs audible pitch glide>
Agent: “That was great. Can you now tap your right forefinger to your right thumb as fast and wide as you can, like this . . . ” <provides a video demonstration>
Responding person: <performs a voice-less finger tap>

In this example the virtual agent 111/AI agent 113 would utilize the speaker 226B to present the comment and question, and the responding person 210A would answer with the audible response and images coming through the combined camera/microphone 224B. The virtual agent/AI agent, in cooperation with the data store 112, would then analyze the semantic cues from the finger movement gestures, and affective content from the pitch glide. In that way, both the semantic content and the affect content would be utilized to provide an assessment of a medical or psychological condition.

FIG. 2C is a perspective view of an assessment session 200C, in which the responding person 210C has schizophrenia. As with FIG. 2A, the question bubble should be interpreted as multiple questions and comments coming from practitioner 120 and/or the virtual agent 111/AI agent 113, and the answer bubble should be interpreted as multiple answers and other audible responses coming from the responding person 210C. A computer 222C operates an optional keyboard 223C, a combined camera/microphone 224C, and a speaker 226C.

Here again, guidance regarding suitable questions and comments can be taken from the priority provisional application, and the relevant literature. Following is an example of a very short portion of a possible assessment.
Agent: “Which of the following topics displayed on your screen would you like to talk about?”
Responding person: “Vacations”.
Agent: “Great, could you tell me more about your vacations?”
Responding person: <proceeds to speak for 2 minutes about vacations>

In this example the virtual agent 111/AI agent 113 would utilize the speaker 226C to present the comment and question, and the responding person 210A would answer with the audible response and images coming through the combined camera/microphone 224C. The virtual agent/AI agent, in cooperation with the data store 112, would then analyze the semantic cues from the spoken language, and affective content from the responsive person exhibiting a still expressionless face and then an emotionally responsive face with brows raised and mouth open. In that way, both the semantic content and the affect content would be utilized to provide an assessment of a medical or psychological condition.

FIG. 2D is a perspective view of an assessment session 200D, in which the responding person 210D has bipolar disorder. As with FIG. 2A, the question bubble should be interpreted as multiple questions and comments coming from practitioner 120 and/or the virtual agent 111/AI agent 113, and the answer bubble should be interpreted as multiple answers and other audible responses coming from the responding person 210D. A computer 222D operates an optional keyboard 223D, a combined camera/microphone 224D, and a speaker 226D.

Here again, guidance regarding suitable questions and comments can be taken from the priority provisional application, and the relevant literature. Following is an example of a very short portion of a possible assessment.
Agent: “Are you planning to do anything this afternoon”?
Responding person: “No. Why bother? Nothing really matters anyway”.
Agent (2 days later): “Are you planning to do anything this afternoon”?
Responding person: “Absolutely. I′m working on an amazing idea that will cure all types of cancer”
Agent: “What is it”?
Responding person: “I haven't figured that out yet, but it's going to be amazing”.

In this example, the virtual agent 111/AI agent 113 would utilize the speaker 226D to present the comment and question, and the responding person 210A would answer with the audible response and images coming through the combined camera/microphone 224D. The virtual agent/AI agent, in cooperation with the data store 112, would then analyze the semantic cues from the spoken language, and affective content from the responsive person exhibiting completely different facial expressions from one day to the next. In that way, both the semantic content and the affect content would be utilized to provide an assessment of a medical or psychological condition.

FIG. 2E is a perspective view of an assessment session 200E, in which the responding person 210E (in this case a child) has autism spectrum disorder. As with FIG. 2A, the question bubble should be interpreted as multiple questions and comments coming from practitioner 120 and/or the virtual agent 111/AI agent 113, and the answer bubble should be interpreted as multiple answers and other audible responses coming from the responding person 210E. A computer 222E operates an optional keyboard 223E, a combined camera/microphone 224E, and a speaker 226E.

Here again, guidance regarding suitable questions and comments can be taken from the priority provisional application, and the relevant literature. Following is an example of a very short portion of a possible assessment.
Agent: “Please look at the following image of a person. How would she say “oh” if she were feeling angry?
Responding person: (angrily): “Oh!”
Agent: “Please describe the following picture. What do you see happening here?”
Responding person: <provides a description of the picture>

In this example, the virtual agent 111/AI agent 113 would utilize the speaker 226E to present the comment and question, and the responding person 210A would answer with the audible response and images coming through the combined camera/microphone 224E. The virtual agent/AI agent, in cooperation with the data store 112, would then use emotion content from the child's speech and facial expression in imitating the semantic and acoustic content of her speech, while describing a picture to form an assessment score. In that way, both the semantic content and the affect content would be utilized to provide an assessment of a medical or psychological condition.

In yet another example, not shown, a practitioner 120 and/or the virtual agent 111/AI agent 113, utilize verbal communication, a camera, and a microphone to assess Amyotrophic Lateral Sclerosis (ALS). As before, guidance regarding suitable questions and comments can be taken from the priority provisional application, and the relevant literature, and following is an example of a very short portion of a possible assessment.

Agent : “Please count up from 1 until you run out of breath”

Responding person: “1 . . . 2 . . . 3 . . . 4 . . . 5 . . . 6 . . . .7 . . . 8 . . . 9 . . . .”

Agent: “Thank you. That was great. Can you now repeat the following sentences after me?”

Responding person: <repeats sentences>

In this example the virtual agent 111/AI agent 113, in cooperation with the data store 112, would use the rate of the responding person's speech to estimate semantic information, the duration of a breath to estimate respiratory information and the facial expression and prosody of speech to estimate affective content.

In the different examples above, there can be differences in the relative importance of audio and video information coming from the responding person. For example, in some examples, hand movements are more important, and in other examples, the speech can be more important. These differences can become significant if there are transmission or other line difficulties. In such cases, the communication agent 114 is configured to make adjustments to prioritize audio over video, or vice versa. This can be done by adjusting the relative bandwidth of audio and video during data streaming and collection, or by using different weighted combinations of content extracted from post-processed audio and video streams in order to produce assessments or inferences.

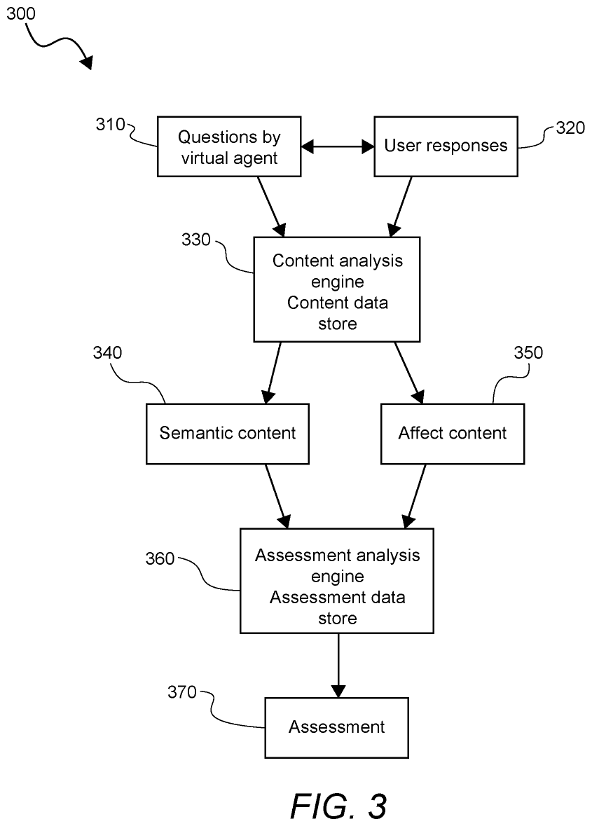

FIG. 3 is a flowchart of a virtual agent and other functionalities of cloud 110 interacting with a responding person, using both semantic content and affect content to assess a condition of the responding person. In block 310 the virtual agent 111/AI agent 113 asks questions to a responding person and provides comments or other guidance. In block 320 the responding person responds in ways that can be perceived through a microphone and camera. In block 330 the virtual agent 111/AI agent 113 interprets the perceived information with respect to semantic content 320 and affect content 350 and utilizes the data store 360 to make an assessment 370.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification refers to at least one of something selected from the group consisting of A, B, C . . . . and N, the text should be interpreted as requiring only one element from the group, not A plus N, or B plus N, etc.

What is claimed is:

1. A method of assessing a medical or psychological condition of a user, comprising configuring a processor to execute instructions that operate a virtual agent configured to:
send questions to the user;
assess audio and image inputs from the user, wherein a later one of the questions branches from an earlier one of the questions, based at least in part upon both (a) semantic content derived from one or more of the audio and image inputs, and (b) affect content derived from one or more of the audio and image inputs; and
apply both the semantic content and the affect content against information in a data store to provide an assessment of the medical or psychological condition.

2. The method of claim 1, wherein the semantic content is derived from a physical characteristic of the user, selected from the group consisting of a facial expression, an eye movement, extent of eye contact, a posture, and a hand gesture.

3. The method of claim 2, wherein the semantic content is derived from a change in the physical characteristic.

4. The method of claim 1, wherein the semantic content is derived from an audible characteristic of speech of the user.

5. The method of claim 4, wherein the audible characteristic is selected from the group consisting of voice pitch, voice speed, voice loudness, and a non-verbal utterance.

6. The method of claim 1, wherein the affect content is derived from a physical characteristic of the user, selected from the group consisting of a facial expression, an eye movement, extent of eye contact, a posture, and a hand gesture.

7. The method of claim 6, wherein the affect content is derived from a change in the physical characteristic.

8. The method of claim 1, wherein the affect content is derived from an audible characteristic of speech of the user.

9. The method of claim 8, wherein the audible characteristic is selected from the group consisting of voice pitch, voice speed, voice loudness, and a non-verbal utterance.

10. The method of claim 1, further comprising a communication agent configured to modify relative bandwidth utilization between the audio and image inputs, based at least in part on at least one of the semantic and affect contents.

11. The method of claim 1, further comprising a communication agent configured to modify relative bandwidth utilization between the audio and image inputs, based at least in part on content of at least one of the questions being asked.

12. The method of claim 1, further comprising a communication agent configured to modify relative bandwidth utilization between the audio and image inputs, based at least in part on rapidity of speech of the user.

13. The method of claim 1, further comprising a communication agent configured to modify relative bandwidth utilization between the audio and image inputs, based at least in part on rapidity of movement of a body part of the user.

14. The method of claim 1, further comprising utilizing an artificial intelligence agent to assist in providing the assessment of the psychological condition by the virtual agent.

15. The method of claim 1, further comprising using (a) the assessment of the psychological condition by the virtual agent and (b) an additional assessment by a human assessor to train an artificial intelligence agent to improve future assessments of psychological conditions of other users.

16. The method of claim 1, wherein the virtual agent is further configured to provide the assessment without direct input from an assessor.

17. The method of claim 1, wherein a branched set of the questions is directed to assessing depression, and the affect content is derived at least in part from a physical characteristic of the user, selected from the group consisting of a facial expression, an eye movement, extent of eye contact, a posture, and a hand gesture.

18. The method of claim 1, wherein a branched set of the questions is directed to assessing bipolar disorder, and the affect content is derived at least in part from a physical characteristic of the user, selected from the group consisting of a facial expression, an eye movement, extent of eye contact, a posture, and a hand gesture.

19. The method of claim 1, wherein a branched set of the questions is directed to assessing Parkinson's disease, and the semantic/affect content is derived at least in part from a physical characteristic of the user, selected from the group consisting of a facial expression, an eye movement, extent of eye contact, a posture, and a hand gesture.

20. The method of claim 1, wherein a branched set of the questions is directed to assessing schizophrenia, and the semantic/affect content is derived at least in part from a physical characteristic of the user, selected from the group consisting of a facial expression, an eye movement, extent of eye contact, a posture, and a hand gesture.

21. The method of claim 1, wherein a branched set of the questions is directed to assessing autism spectrum disorder and the semantic/affect content is derived at least in part from a physical characteristic of the user, selected from the group consisting of a facial expression, an eye movement, extent of eye contact, a posture, and a hand gesture.

22. The method of claim 1, wherein a branched set of the questions is directed to assessing bipolar disorder and the semantic/affect content is derived at least in part from a physical characteristic of the user, selected from the group consisting of a facial expression, an eye movement, extent of eye contact, a posture, and a hand gesture.

23. The method of claim 1, wherein an artificial intelligence agent can simultaneously assist multiple instances of the virtual agent, each of the instances conversing with a different responding person to assess a psychological or other medical condition in parallel.
